(19) United States
(12) Patent Application Publication
(10) Pub. No.: US 2025/0281222 A1
(43) Pub. Date: Sep. 11, 2025

(54) GUIDE WIRE AND MEDICAL SYSTEM

(71) Applicant: ASAHI INTECC CO., LTD., Seto-shi (JP)

(72) Inventors: Naozumi IWATA, Seto-shi (JP); Tomoki ICHIKAWA, Seto-shi (JP); Shoma OKAMOTO, Seto-shi (JP)

(73) Assignee: ASAHI INTECC CO., LTD., Seto-shi (JP)

(21) Appl. No.: 19/213,173

(22) Filed: May 20, 2025

Related U.S. Application Data
(63) Continuation of application No. PCT/JP2022/044157, filed on Nov. 30, 2022.

(51) Int. Cl. A61B 18/04; A61B 18/00

(52) U.S. Cl. CPC A61B 18/042; A61B 2018/00351; A61B 2018/00577; A61B 2018/00595

(57) ABSTRACT

A guide wire includes: a conductive core shaft; a conductive coil body surrounding a distal end portion of the core shaft; a conductive distal tip joined to a distal end of the core shaft and a distal end of the coil body; and an insulating tube made of a resin and covering a proximal end portion of the coil body and a proximal end portion the core shaft. The coil body has: a covered portion covered by the insulating tube; and a protruding portion protruding from a distal end of the insulating tube toward the distal tip, and the protruding portion of the coil body and the distal tip constitute an electrode portion for plasma ablation.

### CROSS-REFERENCE TO RELATED APPLICATIONS

This is a Continuation of Patent Cooperation Treaty Application Number PCT/JP2022/044157 filed Nov. 30, 2022. The disclosure of the prior application(s) is hereby incorporated by reference herein in its entirety.

### TECHNICAL FIELD

The disclosed embodiments relate to a guide wire and a medical system.

### BACKGROUND

As methods for treating arrhythmia resulting in abnormal heartbeat rhythm or chronic total occlusion (CTO) resulting from occlusion of a blood vessel by a lesion, plasma ablation therapies have been recently known, in which a living body tissue is ablated (cauterized) using a plasma flow. For example, Patent Literature 1 discloses a low-temperature plasma-type scalpel device. The device described in Patent Literature 1 includes a transmission electrode and a loop electrode that is inserted into a living body through the same catheter as of the transmission electrode, in which the transmission electrode generates a plasma to vaporize and excise a target by applying a voltage between the transmission electrode and the loop electrode.

### CITATION LIST

### Patent Literature

Patent Literature 1: JP 2021-516138 W

### SUMMARY

### Technical Problem

In the plasma ablation therapies, a plasma guide wire and a guide wire having a return electrode are inserted into a blood vessel, and, in this state, a high-frequency wave is applied to the plasma guide wire and the guide wire from a high-frequency generator. Thereby, a potential difference is generated between a distal end electrode provided on a distal end portion of the plasma guide wire and the return electrode provided on the guide wire, this potential difference causes a streamer corona discharge between the two electrodes. This streamer corona discharge makes it possible to ablate a CTO in the vicinity of the distal end electrode of the plasma guide wire. For correctly generating a plasma on the distal end electrode of the plasma guide wire, the return electrode provided on the guide wire needs to have a surface area larger than of the distal end electrode of the plasma guide wire.

Since a guide wire is inserted into a blood vessel, the guide wire needs to be configured to have a flexible distal end portion to improve safeness. However, in the device described in Patent Literature 1, improvement of the flexibility of the loop electrode on the distal end portion is not taken into account at all. Such problems are common to all guide wires to be inserted into living body lumens such as the lymph gland system, bile tract system, urinary system, respiratory tract system, digestive organ system, secreting gland system, and reproductive organs besides the blood vascular system. Furthermore, it has been required to improve the usability of the medical apparatus.

The disclosed embodiments have been made to solve at least a part of the above-described problems, and an object of the disclosed embodiments is to improve the flexibility of the distal end portion in the guide wire having an electrode portion on its distal end.

### Solution to Problem

The disclosed embodiments have been made to solve at least a part of the above-described problems, and can be embodied as the following aspects.
(1) According to an aspect of the disclosed embodiments, a guide wire is provided. This guide wire includes a conductive core shaft, a conductive coil body surrounding a distal end portion of the core shaft, a conductive distal tip joined to a distal end of the core shaft and a distal end of the coil body, and an insulating tube made of a resin and covering a proximal end portion of the coil body and a proximal end portion of the core shaft. The coil body has a covered portion covered by the insulating tube, and a protruding portion protruding from a distal end of the insulating tube toward the distal tip. The protruding portion of the coil body and the distal tip constitute an electrode portion of the guide wire for plasma ablation.

### BRIEF DESCRIPTION OF DRAWINGS

FIG. 1 is an explanatory view illustrating a configuration of a medical system.

FIG. 2 is an explanatory view illustrating a sectional configuration of a guide wire.

FIG. 3 is an enlarged sectional view illustrating a part of the guide wire on a distal end side.

FIG. 4 is an explanatory view explaining a method of using the medical system.

FIG. 5 is an enlarged sectional view illustrating a part of a guide wire on a distal end side, according to the second embodiment.

FIG. 6 is an enlarged sectional view illustrating a part of a guide wire on a distal end side, according to the third embodiment.

FIG. 7 is an enlarged sectional view illustrating a part of a guide wire on a distal end side, according to the fourth embodiment.

FIG. 8 is an explanatory view illustrating a sectional configuration of a guide wire according to the fifth embodiment.

FIG. 9 is an explanatory view illustrating a sectional configuration of a guide wire according to the sixth embodiment.

### DETAILED DESCRIPTION OF EMBODIMENTS

### First Embodiment

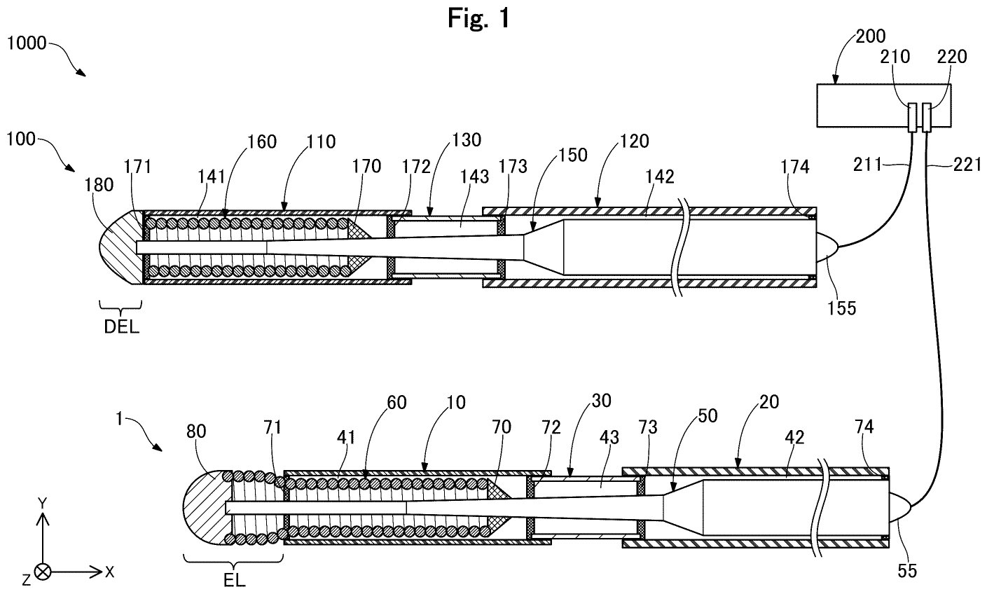

FIG. 1 is an explanatory view illustrating a configuration of a medical system 1000. The medical system 1000 is used for the purpose of recanalizing chronic total occlusion (CTO) or treating mild to moderate stenosis, significant stenosis, arrhythmia, or the like by ablating (cauterizing) a living body tissue using a plasma flow. The medical system 1000 includes a guide wire 1, a plasma guide wire 100, and a radio frequency (RF) generator 200.

The plasma guide wire 100 is a device that has a distal end electrode DEL on its distal end, in which a plasma is generated on the distal end electrode DEL to ablate (cauterize) a target tissue such as CTO. The guide wire 1 is a device that has, on its distal end, an electrode portion EL that functionally serves as a return electrode and is used in combination with the plasma guide wire 100 to generate a plasma on the plasma guide wire 100. The RF generator 200 is an apparatus that applies a high-frequency wave to the plasma guide wire 100 and the guide wire 1. The disclosed embodiments will be explained below with reference to a case where the guide wire 1 and the plasma guide wire 100 are used for recanalizing CTO in a blood vessel, but the guide wire 1 and the plasma guide wire 100 may be inserted not only into vascular systems but also into living body lumens such as lymphatic systems, biliary systems, urinary systems, respiratory systems, digestive systems, secretory glands, and reproductive organs.

The plasma guide wire 100 has an elongated outer shape and includes a distal tip 180, a first tube 110, a second tube 120, a third tube 130, a core shaft 150, a coil body 160, a coil fixation portion 170, a first fixation portion 171, a second fixation portion 172, a third fixation portion 173, and a fourth fixation portion 174.

The distal tip 180 is a conductive member that functionally serves as the distal end electrode DEL for discharging electricity with the electrode portion EL of the guide wire 1. The distal tip 180 is provided on the frontmost end side of the plasma guide wire 100. The distal tip 180 has an outer shape with a diameter gradually decreasing from the proximal end side toward the distal end side for smoothening progress of the plasma guide wire 100 in a blood vessel and for facilitating generation of the plasma. As illustrated in FIG. 1, the distal tip 180 according to the first embodiment has a shape closer to a triangular pyramid (triangular pyramid shape with a roundish tip) compared to a distal tip 80 of the guide wire 1.

The core shaft 150 is a conductive member that constitutes a center axis of the plasma guide wire 100. The core shaft 150 has an elongated outer shape extending in the longitudinal direction of the plasma guide wire 100. The coil body 160 is conductive and surrounds a portion of the core shaft 150 on the distal end side. The coil body 160 is formed by spirally winding a conductive wire. The coil body 160 may be a single thread coil formed by winding one wire in a single thread manner; a multi-thread coil formed by winding a plurality of wires in a multiple thread manner; a single thread twisted wire coil formed by winding, in a single thread manner, a twisted wire with a plurality of wires twisted; or a multi-thread twisted wire coil formed by winding, in a multiple thread manner, each of a plurality of twisted wires with a plurality of wires twisted.

The first tube 110, the second tube 120, and the third tube 130 are all a hollow cylindrical tubular body made of an insulating resin. The first tube 110 is provided on the proximal end side with respect to the distal tip 180 to cover the distal end side of the core shaft 150 and the coil body 160. The second tube 120 is provided on the proximal end side with respect to the third tube 130 to cover the proximal end side of the core shaft 150. The third tube 130 is provided between the first tube 110 and the second tube 120 to cover an intermediate portion of the core shaft 150. A distal end portion of the third tube 130 is joined to a proximal end portion of the first tube 110. A proximal end portion of the third tube 130 is joined to a distal end portion of the second tube 120. The third tube 130 has an outer diameter smaller than of the first tube 110 and smaller than of the second tube 120. The third tube 130 has a distal end portion overlapped with the proximal end portion of the first tube 110, and a proximal end portion overlapped with the distal end portion of the second tube 120.

The first tube 110 has a gas layer 141 filled with a gas between the first tube 110 and the core shaft 150/coil body 160. The second tube 120 has a gas layer 142 filled with a gas between the second tube 120 and the core shaft 150. The third tube 130 has a gas layer 143 filled with a gas between the third tube 130 and the core shaft 150. As the gas constituting the gas layers 141, 142, and 143, it is possible to use air, sulfur hexafluoride (SF₆) gas, or hydrogen (H₂) gas. When air is used as the gas, the gas layers 141, 142, and 143 may also be referred to as air layers 141, 142, and 143, respectively.

The coil fixation portion 170 is a member that fixes the proximal end portion of the coil body 160 and a part of the core shaft 150. The first fixation portion 171 is a member that is provided on the distal end portion of the first tube 110 to fix the distal end portion of the first tube 110, the distal end portion of the core shaft 150, and the distal end portion of the coil body 160. The second fixation portion 172 is a member that is provided on the distal end portion of the third tube 130 to fix the distal end portion of the third tube 130, the proximal end portion of the first tube 110, and a part of the core shaft 150. The third fixation portion 173 is a member that is provided on the proximal end portion of the third tube 130 to fix the proximal end portion of the third tube 130, the distal end portion of the second tube 120, and a part of the core shaft 150. The fourth fixation portion 174 is a member that is provided on the proximal end portion of the second tube 120 to fix the proximal end portion of the second tube 120 and the proximal end portion of the core shaft 150.

The core shaft 150 and the distal tip 180 can be made of any conductive material, such as a chromium-molybdenum steel, a nickel-chromium-molybdenum steel, a stainless steel such as SUS304, and a nickel-titanium alloy. The distal tip 80 may be formed by melting the distal end portion of the core shaft 150 with a laser or the like. The first tube 110, the second tube 120, and the third tube 130 can be made of any insulating material, e.g. a copolymer of tetrafluoroethylene and perfluoroalkoxy ethylene (PFA); a polyolefin such as polyethylene, polypropylene, and ethylene-propylene copolymer; a polyester such as polyethylene terephthalate; polyvinyl chloride; an ethylene-vinyl acetate copolymer; a crosslinked ethylene-vinyl acetate copolymer; a thermoplastic resin such as polyurethane; a polyamide elastomer; a polyolefin elastomer; a silicone rubber; a latex rubber; and a super engineering plastic such as polyetheretherketone, polyetherimide, polyamide-imide, polysulfone, polyimide, and polyethersulfone. Each of the first tube 110, the second tube 120, and the third tube 130 may be made of a same material, or may be made of different materials. The coil fixation portion 170, the first fixation portion 171, the second fixation portion 172, the third fixation portion 173, and the fourth fixation portion 174 may be made of any bonding agent such as an epoxy adhesive.

The RF generator 200 outputs a high frequency power to between a first terminal 210 and a second terminal 220. From the first terminal 210, a first cable 211 extends. The first cable 211 is connected to a proximal end portion 155 of the plasma guide wire 100. From the second terminal 220, a second cable 221 extends. The second cable 221 is connected to the proximal end portion 55 of the guide wire 1. The first cable 211 and the second cable 221 are conductive electric wires. The first cable 211 and the second cable 221 may have a cable connector (connection terminal for physical and electrical connection between cables). The RF generator 200 functionally serves as a “high-frequency generator”.

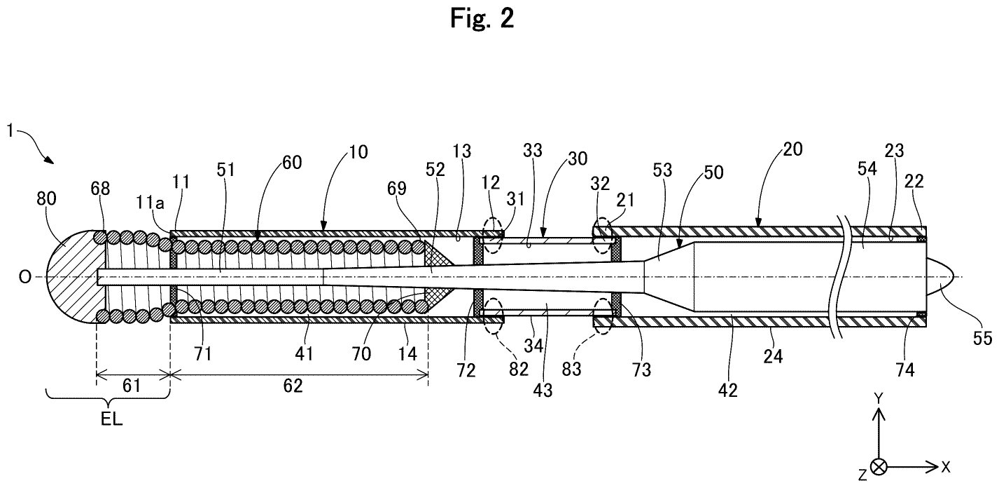

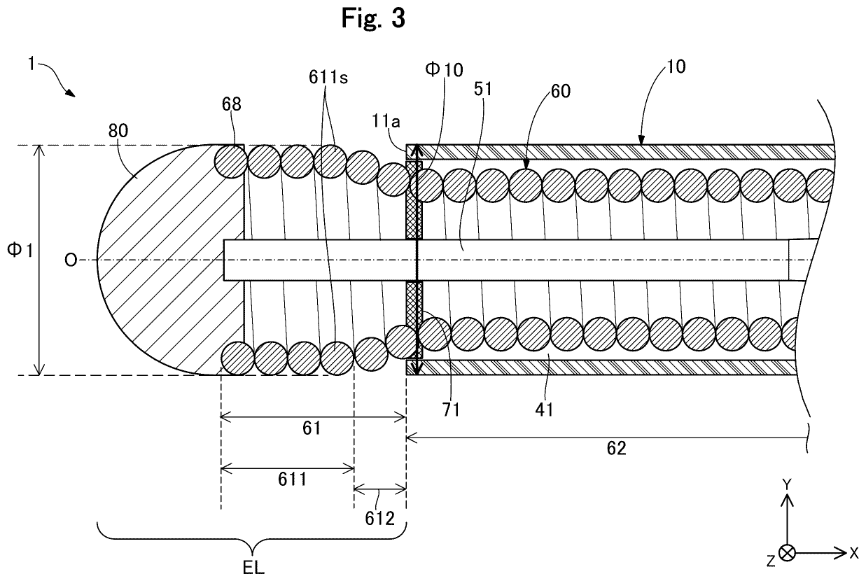

FIG. 2 is an explanatory view illustrating a sectional configuration of the guide wire 1. FIG. 3 is an enlarged sectional view illustrating a part of the guide wire 1 on the distal end side. FIG. 2 and FIG. 3 are sectional views taken along the longitudinal section, as in FIG. 1. The configuration of the guide wire 1 will be explained below with reference to FIG. 1 to FIG. 3. As described above, the guide wire 1 has, on its distal end, the electrode portion EL that functionally serves as a return electrode, and used in combination with the plasma guide wire 100 to generate a plasma on the plasma guide wire 100.

In FIG. 2 and FIG. 3, an axis passing through the center of the guide wire 1 is expressed by an axis line O (dashed-dotted line). In the examples of FIG. 2 and FIG. 3, the axis line O coincides with the axis passing through the center of each constituent member of the guide wire 1, i.e. a first tube 10, a second tube 20, a third tube 30, the distal tip 80, a core shaft 50, and a coil body 60. However, the axis line O may be inconsistent with the center axis of each constituent member in the guide wire 1. In FIG. 2 and FIG. 3, XYZ axes orthogonal to each other are illustrated. The X axis corresponds to the longitudinal direction of the guide wire 1, the Y axis corresponds to the height direction of the guide wire 1, and the Z axis corresponds to the width direction of the guide wire 1. The left side (−X axis direction) in FIG. 2 and FIG. 3 is referred to as “distal end side” of the guide wire 1 and each constituent member, and the right side (+X axis direction) in FIG. 2 and FIG. 3 is referred to as “proximal end side” of the guide wire 1 and each constituent member. Of both ends in the longitudinal direction (X axis direction), one end located on the distal end side is referred to as “distal end”, and the other end located on the proximal end side is referred to as “proximal end”. The distal end and its vicinity are referred to as “distal end portion”, and the proximal end and its vicinity are referred to as “proximal end portion”. The distal end side is inserted into a living body, and the proximal end side is operated by an operator such as a surgeon. The same applies to the figures following FIG. 2 and FIG. 3.

The guide wire 1 has an elongated outer shape and includes the first tube 10, the second tube 20, the third tube 30, the core shaft 50, the distal tip 80, the coil body 60, a coil fixation portion 70, a first fixation portion 71, a second fixation portion 72, a third fixation portion 73, a fourth fixation portion 74.

The distal tip 80 is conductive member that discharges electricity on the distal tip 180 of the plasma guide wire 100 used in combination with the guide wire 1 (FIG. 1). The distal tip 80 is provided on the frontmost end side of the guide wire 1 (i.e. the distal end portion of the guide wire 1). The distal tip 80 has an outer shape with a diameter gradually decreasing from the proximal end side toward the distal end side for smoothening progress of the guide wire 1 in a blood vessel. As illustrated in FIG. 2 and FIG. 3, the distal tip 80 according to the first embodiment is hemispherical. The maximum outer diameter of the distal tip 80 (i.e. the outer diameter of the proximal end portion of the distal tip 80) is substantially equal to an outer diameter Φ1 of a protruding portion 61 described later. The proximal end portion of the distal tip 80 is joined to the distal end of the core shaft 50 and a distal end 68 of the coil body 60. Any bonding agent such as an epoxy adhesive can be used for the joining. Further, laser welding or the like may be used as a joining means.

The core shaft 50 is a conductive member that constitutes the center axis of the guide wire 1. The core shaft 50 has an elongated outer shape extending in the longitudinal direction of the guide wire 1. The core shaft 50 includes a small diameter portion 51, a first tapered portion 52, a second tapered portion 53, and a large diameter portion 54 in this order from the distal end toward the proximal end. The small diameter portion 51 is a portion where the outer diameter of the core shaft 50 is the smallest, and has a substantially columnar shape with a constant outer diameter from the distal end to the proximal end. The first tapered portion 52 is provided between the small diameter portion 51 and the second tapered portion 53 and has an outer shape with a diameter gradually decreasing from the proximal end side toward the distal end side. The second tapered portion 53 is provided between the first tapered portion 52 and the large diameter portion 54 and has an outer shape with an outer diameter gradually decreasing from the proximal end side toward the distal end side at an inclination angle different from that of the first tapered portion 52. The large diameter portion 54 is a portion where the outer diameter of the core shaft 50 is the largest and has a substantially columnar shape with a constant outer diameter from the distal end to the proximal end. The proximal end portion 55 of the large diameter portion 54 is a portion where a proximal end surface of the large diameter portion 54 bulges.

In the first embodiment, the “constant” is synonymous with “substantially constant”, and means that the diameter is substantially constant while accepting fluctuation due to production errors or the like. In the first embodiment, when the transverse section of the member (or the inner cavity) is elliptical, the “outer diameter” and the “inner diameter” refer to the length of the largest diameter in any transverse section.

The coil body 60 is conductive and arranged so as to surround a part of the core shaft 50 on the distal end side. In the example of FIG. 2, the coil body 60 surrounds, in the core shaft 50, the small diameter portion 51 and a part of the first tapered portion 52 on the distal end side. The coil body 60 is formed by spirally winding a conductive wire. The coil body 60 may be a single thread coil formed by winding one wire in a single thread manner; a multi-thread coil formed by winding a plurality of wires in a multiple thread manner; a single thread twisted wire coil formed by winding, in a single thread manner, a twisted wire with a plurality of wires twisted; or a multi-thread twisted wire coil formed by winding, in a multiple thread manner, each of a plurality of twisted wires with a plurality of wires twisted.

As illustrated in FIG. 2, the coil body 60 has a protruding portion 61 and a covered portion 62. The covered portion 62 is a portion of the coil body 60, which is covered by the insulating tubes 10, 20, and 30 (specifically, the first tube 10). The protruding portion 61 is a portion of the coil body 60, which is not covered by the insulating tubes 10, 20, and 30 but protrudes from a distal end 11a of the insulating tubes 10, 20, and 30 (specifically, first tube 10) toward the distal tip 80 side. In other words, it can be said that the protruding portion 61 is a portion of the coil body 60, which protrudes from the distal end 11a of the first tube 10 toward the distal end side (in the −X axis direction).

As illustrated in FIG. 3, the protruding portion 61 of the coil body 60 has a straight portion 611 and a tapered portion 612. The straight portion 611 is a portion of the protruding portion 61, which has a constant outer diameter Φ1. The straight portion 611 is provided on the frontmost end side of the coil body 60 (i.e. on the distal end side with respect to the tapered portion 612). The tapered portion 612 is a portion of the protruding portion 61, which is provided on the proximal end side with respect to the straight portion 611 and has an outer diameter gradually decreasing from the distal end toward the proximal end. In the first embodiment, the “outer diameter” of the protruding portion 61, the covered portion 62, the straight portion 611, and the tapered portion 612 refers to the outer diameter of the thickest portion of the wire constituting each portion.

The first tube 10, the second tube 20, and the third tube 30 are all hollow cylindrical tubular bodies made of an insulating resin. The first tube 10, the second tube 20, and the third tube 30 are also collectively referred to as “insulating tubes 10, 20, and 30”.

The first tube 10 is provided on the proximal end side with respect to the distal tip 80 and the protruding portion 61 and on the distal end side with respect to the second tube 20 and the third tube 30. The first tube 10 covers the covered portion 62 of the coil body 60 and a part of the core shaft 50 (specifically, a part of the small diameter portion 51 on the proximal end side and a part of the first tapered portion 52 on the distal end side). The inner diameter of the first tube 10 is larger than the outer diameter of the covered portion 62. The thickness and length of the first tube 10 may be arbitrarily determined. The second tube 20 is provided on the proximal end side with respect to the first tube 10 and the second tube 20. The second tube 20 covers the proximal end portion of the first tapered portion 52 of the core shaft 50, the second tapered portion 53, and the large diameter portion 54. The proximal end portion 55 of the large diameter portion 54 is not covered by the second tube 20 but is exposed to the outside. The second tube 20 has an inner diameter larger than the outer diameter of the large diameter portion 54 of the core shaft 50. A thickness and a length of the second tube 20 may be arbitrarily determined.

The third tube 30 is provided between the first tube 10 and the second tube 20. The third tube 30 covers an intermediate portion of the first tapered portion 52 of the core shaft 50. A thickness and a length of the third tube 30 may be arbitrarily determined. As illustrated in FIG. 2, the distal end portion 31 of the third tube 30 is joined to a proximal end portion 12 of the first tube 10. A proximal end portion 32 of the third tube 30 is joined to a distal end portion 21 of the second tube 20. The third tube 30 has an outer diameter smaller than of the first tube 10 and smaller than of the second tube 20. Further, as illustrated in FIG. 2, the third tube 30 is arranged such that the distal end portion 31 of the third tube 30 is overlapped with the proximal end portion 12 of the first tube 10 and the proximal end portion 32 of the third tube 30 is overlapped with the distal end portion 21 of the second tube 20. Thus, in the third tube 30, an outer peripheral surface 34 of the distal end portion 31 is joined to an inner peripheral surface 13 of the proximal end portion 12 of the first tube 10 and the outer peripheral surface 34 of the proximal end portion 32 is joined to an inner peripheral surface 23 of the distal end portion 21 of the second tube 20. A portion of the third tube 30, which is located between the distal end portion 31 and the proximal end portion 32 (intermediate portion) is not covered with the first tube 10 or the second tube 20 but is exposed to the outside.

For the joining of the first tube 10, the second tube 20, and the third tube 30, any bonding agent such as an epoxy adhesive can be used. In FIG. 2, the joint portion between the third tube 30 and the first tube 10 is illustrated as a distal end side joint part 82 (dashed circle frame), and the joint portion between the third tube 30 and the second tube 20 is illustrated as a proximal end side joint part 83 (dashed circle frame). As illustrated in FIG. 2, the insulating tubes 10, 20, and 30 according to the first embodiment have a constricted shape on the intermediate portion where the third tube 30 is provided.

As illustrated in FIG. 2, the first tube 10 has a gas layer 41 filled with a gas between the inner peripheral surface 13 of the first tube 10 and the outer peripheral surfaces of the core shaft 50/covered portion 62. The gas layer 41 is provided entirely in the circumferential direction. The gas layer 41 is provided entirely in the longitudinal direction from the distal end portion 11 to the proximal end portion 12 of the first tube 10 (specifically, entirely in the longitudinal direction from the proximal end of the first fixation portion 71 to the distal end of the second fixation portion 72) excluding sites where the first fixation portion 71 and the second fixation portion 72 are provided. The second tube 20 has a gas layer 42 filled with a gas between the inner peripheral surface 23 of the second tube 20 and the outer peripheral surface of the core shaft 50. Similarly to the gas layer 41, the gas layer 42 is provided entirely in the circumferential direction. The gas layer 42 is provided entirely in the longitudinal direction from the distal end portion 21 to the proximal end portion 22 of the second tube 20 (specifically, entirely in the longitudinal direction from the proximal end of the third fixation portion 73 to the distal end of the fourth fixation portion 74) excluding sites where the third fixation portion 73 and the fourth fixation portion 74 are provided. The third tube 30 has a gas layer 43 filled with a gas between an inner peripheral surface 33 of the third tube 30 and the outer peripheral surface of the core shaft 50. Similarly to the gas layer 41, the gas layer 43 is provided entirely in the circumferential direction. The gas layer 43 is provided entirely in the longitudinal direction from the distal end portion 31 to the proximal end portion 32 of the third tube 30 (specifically, entirely in the longitudinal direction from the proximal end of the second fixation portion 72 to the distal end of the third fixation portion 73) excluding sites where the second fixation portion 72 and the third fixation portion 73 are provided.

As the gas constituting the gas layers 41, 42, and 43, any gas may be used as long as the gas is more electrically insulative than the insulating resin constituting the first, second, and third tubes 10, 20, and 30. As the gas constituting the gas layers 41, 42, and 43, for example, air, sulfur hexafluoride (SF₆) gas, or hydrogen (H₂) gas may be used. When air is used as the gas, the gas layers 41, 42, and 43 may also be referred to as air layers 41, 42, and 43.

The coil fixation portion 70 fixes a proximal end 69 of the covered portion 62 of the coil body 60 and a part of the first tapered portion 52 of the core shaft 50. The first fixation portion 71 is provided on the distal end portion 11 of the first tube 10 to fix the distal end portion 11 of the first tube 10, a part of the coil body 60 (specifically, a boundary portion between the protruding portion 61 and the covered portion 62), and a part of the small diameter portion 51 of the core shaft 50. The first fixation portion 71 is provided entirely in the circumferential direction to block the gas flow inside and outside of the guide wire 1 (specifically, the flow of the gas constituting the gas layer 41).

The second fixation portion 72 is provided on the distal end portion 31 of the third tube 30 to fix the distal end portion 31 of the third tube 30, the proximal end portion 12 of the first tube 10, and a part of the first tapered portion 52 of the core shaft 50. The second fixation portion 72 is provided entirely in the circumferential direction to block the gas flow between the gas layer 41 and the gas layer 43. The third fixation portion 73 is provided on the proximal end portion 32 of the third tube 30 to fix the proximal end portion 32 of the third tube 30, the distal end portion 21 of the second tube 20, and a part of the first tapered portion 52 of the core shaft 50. The third fixation portion 73 is provided entirely in the circumferential direction to block the gas flow between the gas layer 43 and the gas layer 42. The fourth fixation portion 74 is provided on the proximal end portion 22 of the second tube 20 to fix the proximal end portion 22 of the second tube 20 and the proximal end portion of the large diameter portion 54 of the core shaft 50. The fourth fixation portion 74 is provided entirely in the circumferential direction to block the gas flow inside and outside of the guide wire 1 (specifically, the flow of the gas constituting the gas layer 42).

The core shaft 50 and the distal tip 80 can be made of any conductive material, such as a chromium-molybdenum steel, a nickel-chromium-molybdenum steel, a stainless steel such as SUS304, and a nickel-titanium alloy. The distal tip 80 may be formed by melting the distal end portion of the core shaft 50 with a laser or the like.

The first tube 10, the second tube 20, and the third tube 30 can be made of any insulating material, e.g. a copolymer of tetrafluoroethylene and perfluoroalkoxy ethylene (PFA); a polyolefin such as polyethylene, polypropylene, and ethylene-propylene copolymer; a polyester such as polyethylene terephthalate; polyvinyl chloride; an ethylene-vinyl acetate copolymer; a crosslinked ethylene-vinyl acetate copolymer; a thermoplastic resin such as polyurethane; a polyamide elastomer; a polyolefin elastomer; a silicone rubber; a latex rubber; and a super engineering plastic such as polyetheretherketone, polyetherimide, polyamide-imide, polysulfone, polyimide, and polyethersulfone. Each of the first tube 10, the second tube 20, and the third tube 30 may be made of a same material, or may be made of different materials depending on the performance required for the guide wire 1 (e.g. flexibility, torquability, and shape maintainability of the distal end portion).

The coil fixation portion 70, the first fixation portion 71, the second fixation portion 72, the third fixation portion 73, and the fourth fixation portion 74 can be made of any bonding agent such as an epoxy adhesive.

As described above, the coil body 60 and the distal tip 80 are electrically conductive, and meanwhile the insulating tubes 10, 20, and 30 are electrically insulative. As illustrated in FIG. 3, in the guide wire 1 according to the first embodiment, the protruding portion 61 of the coil body 60 protrudes from the distal end 11a of the insulating tube 10, 20, and 30 (specifically, the first tube 10) toward the distal tip 80 side. As a result, the protruding portion 61 is not covered by the insulating tubes 10, 20, and 30 but exposed to the outside, and therefore can functionally serve as the electrode portion EL together with the distal tip 80. The electrode portion EL is used as a return electrode for the distal end electrode DEL (FIG. 1) of the plasma guide wire 100, i.e. an electrode for plasma ablation. The surface of the wire constituting the protruding portion 61 may be coated with a conductive resin or the like. Also in this case, the protruding portion 61 is not covered by the insulating tubes 10, 20, and 30 but is exposed, and therefore can functionally serve as the electrode portion EL.

For correctly generating a plasma on the distal end electrode DEL of the plasma guide wire 100, the surface area of the electrode portion EL provided on the guide wire 1 needs to be larger than the surface area of the distal end electrode DEL of the plasma guide wire 100. In this regard, according to the configuration explained in FIG. 2 and FIG. 3, the surface area of the electrode portion EL is a sum of the surface area of the protruding portion 61 and the surface area of the distal tip 80. In other words, the surface area of the protruding portion 61 can be added to the surface area of the electrode portion EL.

As illustrated in FIG. 3, since the straight portion 611 of the protruding portion 61 has a constant outer diameter Φ1, the outer diameter of the proximal end of the straight portion 611 is also Φ1. In the guide wire 1 according to the first embodiment, the outer diameter Φ1 of the proximal end of the straight portion 611 is equal to the outer diameter Φ10 of the distal end of the insulating tubes 10, 20, and 30 (specifically, outer diameter Φ10 of the distal end 11a of the first tube 10). Herein, the terms “the same” and “equal” include not only a meaning of strict conformity but also a meaning of allowing differences due to production errors or the like. In FIG. 3, for convenience of illustration, the arrow indicating the outer diameter Φ10 is illustrated slightly on the proximal end side with respect to the distal end 11a of the first tube 10.

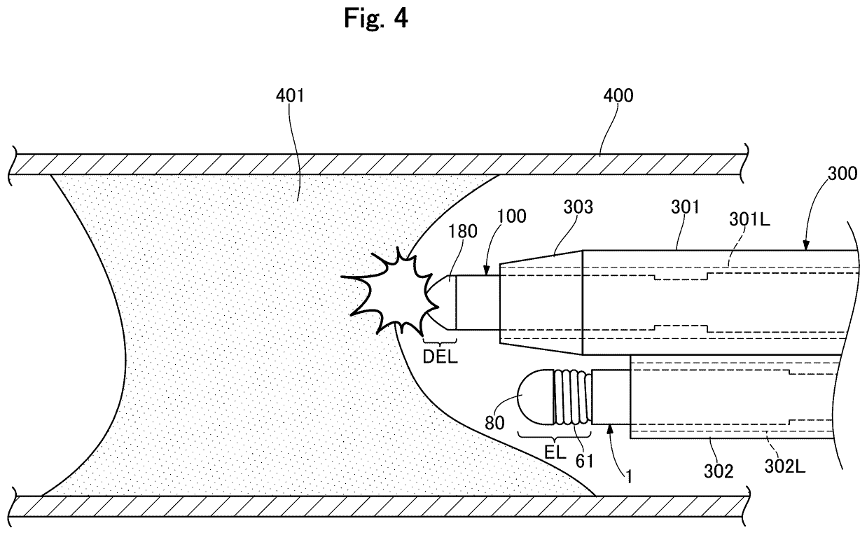

FIG. 4 is a diagram illustrating the method of using the medical system 1000. First, an operator inserts a delivery guide wire into a blood vessel 400 and delivers the delivery guide wire to the vicinity of a CTO401. The operator inserts the delivery guide wire into a catheter 300 and delivers the catheter 300 along the delivery guide wire to the vicinity of the CTO401. In FIG. 4, the catheter 300 is exemplified by a so-called multilumen catheter including a first shaft 301 having a first lumen 301L, a second shaft 302 having a second lumen 302L, and a distal tip 303. The operator inserts the plasma guide wire 100 into the first lumen 301L and protrudes the distal end electrode DEL of the plasma guide wire 100 to the outside from a distal end opening of the first lumen 301L to place the distal end electrode DEL on the vicinity of a tissue to be ablated (CTO401). Similarly, the operator inserts the guide wire 1 into the second shaft 302 and protrudes the electrode portion EL of the guide wire 1 to the outside from the distal end opening of the second lumen 302L. In this state, the operator outputs a high-frequency power from the RF generator 200. Then, due to a potential difference between the distal end electrode DEL of the plasma guide wire 100 and the electrode portion EL of the guide wire 1, streamer corona discharge occurs between the distal end electrode DEL and the electrode portion EL. This streamer corona discharge makes it possible to ablate the CTO in the vicinity of the distal end electrode DEL of the plasma guide wire 100, as illustrated in FIG. 4.

Although a case where the guide wire 1 and the plasma guide wire 100 is delivered using the catheter 300 as the multilumen catheter has been described as an example in FIG. 4, the guide wire 1 and the plasma guide wire 100 may be delivered without using the catheter 300. The guide wire 1 and the plasma guide wire 100 may be individually delivered using two different catheters. Although a case where another delivery guide wire is used for delivering the catheter 300 has been described as an example in FIG. 4, the guide wire 1 or the plasma guide wire 100 may be used as a delivery guide wire. Although a case where the CTO401 is ablated from a true lumen of the blood vessel 400 has been described as an example in FIG. 4, it is possible to adopt a process in which a guide wire is once proceeded from the true lumen of the blood vessel 400 to a false lumen, then the end of the CTO 401 is ablated from the false lumen, and the guide wire is proceeded to the true lumen. In this case, it is possible to place both the guide wire 1 and the plasma guide wire 100 in the false lumen, or alternatively it is possible to place only the plasma guide wire 100 in the false lumen and to place the guide wire 1 in the true lumen. Furthermore, although a case where the distal end electrode DEL of the plasma guide wire 100 is located on the distal end side with respect to the electrode portion EL in the blood vessel 400 has been described as an example in FIG. 4, this positional relationship between the two electrodes may be reversed, or the two electrodes may be at the same position. As described above, the guide wire 1 and the plasma guide wire 100 can be used in any way and in combination of any devices.

As described above, in the guide wire 1 according to the first embodiment, the coil body 60 has the protruding portion 61 that is not covered by the insulating tubes 10, 20, and 30 but protrudes from the distal end 11a of the insulating tubes 10, 20, and 30 toward the distal tip 80 side, in which the protruding portion 61 of the coil body 60 and the distal tip 80 constitute the electrode portion EL for plasma ablation. That means, in the guide wire 1 according to the first embodiment, since the surface area of the electrode portion EL is a sum of the surface area of the protruding portion 61 of the coil body 60 and the surface area of the distal tip 80 (i.e. the surface area of the protruding portion 61 of the coil body 60 can be added to the surface area of the electrode portion EL), the surface area of the electrode portion EL can be easily increased compared to the conventional configurations with an electrode portion consisting only of a distal tip. If the electrode portion consists only of the distal tip, the distal tip needs to be enlarged for increasing the surface area of the electrode portion, which may compromise the flexibility of the distal end portion of the guide wire. However, in the guide wire 1 according to the first embodiment, since the surface area of the electrode portion EL is a sum of the surface area of the protruding portion 61 of the coil body 60 and the surface area of the distal tip 80, the distal tip 80 need not be excessively enlarged, and there is no risk of compromising the flexibility of the distal end portion in the guide wire 1. Furthermore, the protruding portion 61 has a coil shape, and therefore, even if the distal end portion (electrode portion EL) of the guide wire 1 collides with a blood vessel wall, the impact can be relieved by the protruding portion 61, and damage to the blood vessel wall can be suppressed. As a result, in the guide wire 1 having the electrode portion EL on its distal end, the flexibility of the distal end portion and the safeness of the procedure can be improved.

In the guide wire 1 according to the first embodiment, since the protruding portion 61 of the coil body 60 has the straight portion 611 having a constant outer diameter Φ1, and the tapered portion 612 provided on the proximal end side with respect to the straight portion 611 and having an outer diameter gradually decreasing from the distal end to the proximal end, the surface area of the protruding portion 61 can be increased while the outer diameter Φ1 of the protruding portion 61 is maintained constant, i.e. the surface area of the electrode portion EL can be increased. Since the large surface area of the electrode portion EL can contribute not only to correct plasma generation on the distal end electrode DEL of the plasma guide wire 100 used in combination with the guide wire 1 but also to improvement in visibility of the electrode portion EL in an X-ray image (angiogram), the usability of the guide wire 1 can be improved. Also, the increase in the surface area of the electrode portion EL leads to reduction in the risk of blood vessel perforation, and therefore the safeness of the guide wire 1 can be improved. Since the protruding portion 61 of the coil body 60 has, on the proximal end side with respect to the straight portion 611, the tapered portion 612 having the outer diameter gradually decreasing from the distal end toward the proximal end, a rigidity of the protruding portion 61 can be gradually changed, and as a result, fracture of the protruding portion 61 due to a rigidity gap can be suppressed.

Furthermore, in the guide wire 1 according to the first embodiment, since the outer diameter Φ1 of the proximal end of the straight portion 611 is equal to the outer diameters Φ10 of the distal ends of the insulating tubes 10, 20, and 30 (specifically, first tube 10), the outer diameter of the entire distal end side (specifically, the electrode portion EL excluding the tapered portion 612, and the first tube 10) of the guide wire 1 can be made constant. As a result, the guide wire 1 can be prevented from being caught in the blood vessel or caught by other devices (e.g. plasma guide wire 100, and catheter 300 in FIG. 4).

Furthermore, in the guide wire 1 according to the first embodiment, since the electrode portion EL is a return electrode, the guide wire 1 can be configured as a so-called return guide wire that is used in combination with the plasma guide wire 100.

### Second Embodiment

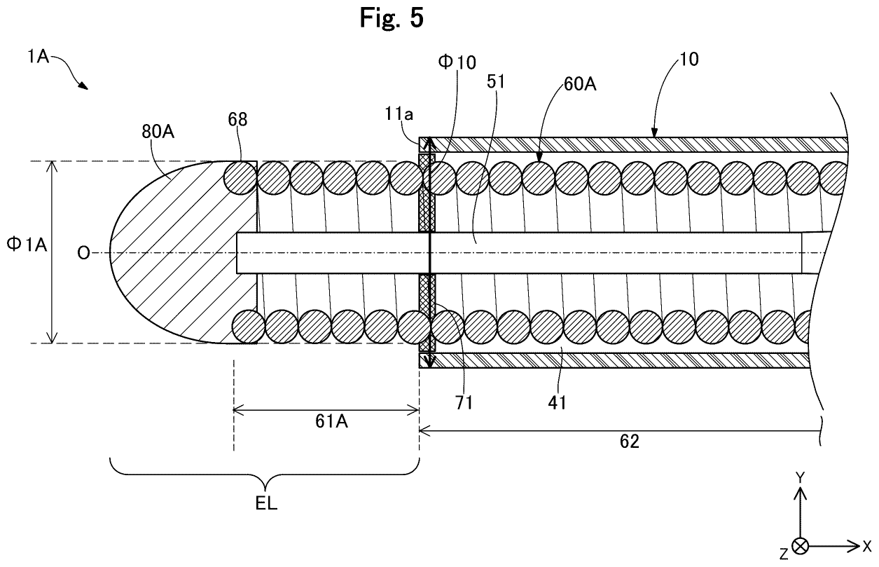

FIG. 5 is an enlarged sectional view illustrating a part of a guide wire 1A on a distal end side, according to the second embodiment. The guide wire 1A according to the second embodiment includes a coil body 60A instead of the coil body 60 and a distal tip 80A instead of the distal tip 80 in the configuration described in the first embodiment.

The coil body 60A includes a protruding portion 61A instead of the protruding portion 61. The protruding portion 61A does not include the straight portion 611 and the tapered portion 612 described in the first embodiment, and has a straight shape with an outer diameter Φ1A constant in whole. The outer diameter Φ1A of the protruding portion 61A is equal to the outer diameter of the covered portion 62. That means, the coil body 60A has a constant outer diameter from the distal end to the proximal end. The outer diameter Φ1A of the protruding portion 61A is smaller than the outer diameter Φ10 of the distal ends of the insulating tubes 10, 20, and 30 (specifically, the outer diameter Φ10 of the distal end 11a of the first tube 10). The maximum outer diameter of the distal tip 80A (i.e. the outer diameter of the proximal end portion of the distal tip 80A) is substantially equal to the outer diameter Φ1A of the protruding portion 61A.

As described above, the configuration of the coil body 60A can be variously modified, and the coil body 60A may be configured so as to have a constant outer diameter from the distal end to the proximal end. Also, the guide wire 1A according to the second embodiment as described above can exhibit the same effect as in the aforementioned first embodiment. According to the configuration of the second embodiment, the process for the guide wire 1 can be simplified.

### Third Embodiment

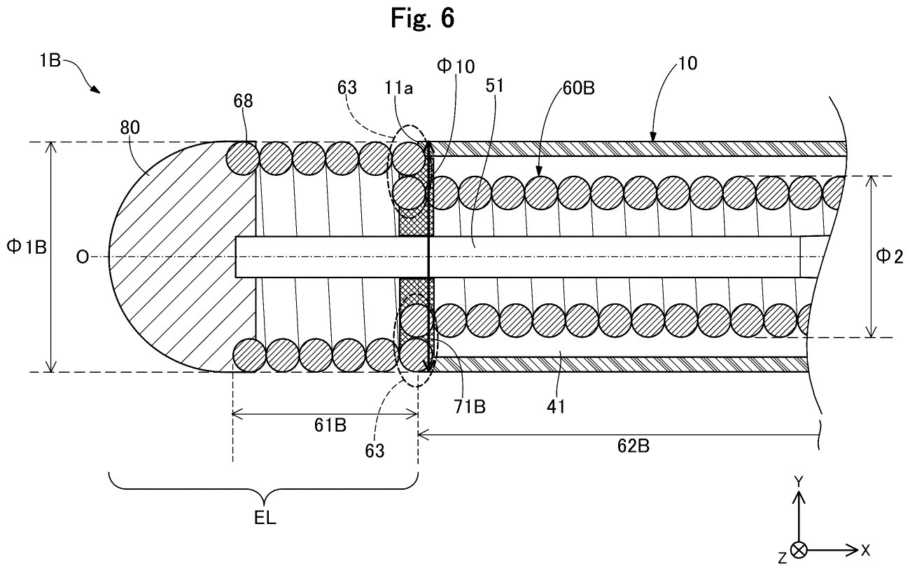

FIG. 6 is an enlarged sectional view illustrating a part of a guide wire 1B on a distal end side, according to the third embodiment. The guide wire 1B according to the third embodiment includes a coil body 60B instead of the coil body 60 and a first fixation portion 71B instead of the first fixation portion 71 in the configuration described in the first embodiment.

The coil body 60B includes a protruding portion 61B instead of the protruding portion 61 and a covered portion 62B instead of the covered portion 62, as well as a stepped portion 63. The protruding portion 61B has a straight shape with an outer diameter Φ1B constant in whole. The covered portion 62B has a straight shape with an outer diameter Φ2 constant in whole. The outer diameter Φ1B of the protruding portion 61B corresponds to the “first outer diameter”, and the outer diameter Φ2 of the covered portion 62B corresponds to the “second outer diameter”. The outer diameter Φ2 (second outer diameter) of the covered portion 62B is smaller than the outer diameter Φ1B (first outer diameter) of the protruding portion 61B. The stepped portion 63 is provided between the protruding portion 61B and the covered portion 62B, where the outer diameter of the coil body 60B changes from the first outer diameter Φ1B to the second outer diameter Φ2. As illustrated in FIG. 6, the protruding portion 61B and the covered portion 62B extend along the longitudinal direction (axial direction) of the guide wire 1B, and meanwhile the stepped portion 63 extends along the circumferential direction of the guide wire 1B.

As illustrated in FIG. 6, since the protruding portion 61B has a constant outer diameter Φ1B, the outer diameter of the proximal end of the protruding portion 61B is also Φ1B. In the guide wire 1B according to the third embodiment, the outer diameter 1B of the proximal end of the protruding portion 61B is equal to the outer diameter Φ10 of the distal ends of the insulating tubes 10, 20, and 30 (specifically, the outer diameter Φ10 of the distal end 11a of the first tube 10). The first fixation portion 71B is provided on the distal end portion 11 of the first tube 10 to fix the distal end portion 11 of the first tube 10 and the stepped portion 63 of the coil body 60B.

As described above, the configuration of the coil body 60B can be variously modified, and the coil body 60B may be configured such that the protruding portion 61B and the covered portion 62B each have a constant outer diameter, and a stepped portion 63 is provided between the protruding portion 61B and the covered portion 62B. Also, the guide wire 1B according to the third embodiment as described above can exhibit the same effect as in the aforementioned first embodiment.

According to the configuration of the third embodiment, the protruding portion 61B has a constant first outer diameter Φ1B, and the first outer diameter Φ1B of the protruding portion 61B is larger than the second outer diameter Φ2 of the covered portion 62B. Thereby, the surface area of the protruding portion 61B can be increased, i.e. the surface area of the electrode portion EL can be increased. As a result, the usability and safeness of the guide wire 1B can be further improved. In the guide wire 1B according to the third embodiment, since the outer diameter Φ1B of the proximal end of the protruding portion 61B is equal to the outer diameter Φ10 of the distal end 11a of the first tube 10, the outer diameter of the entire distal end side of the guide wire 1B (specifically, the electrode portion EL and the first tube 10) can be made constant. As a result, the guide wire 1B can be prevented from being caught in the blood vessel or caught by other devices used in combination. Furthermore, fracture of the coil body 60B due to the step on the outer surface of the guide wire 1B on the distal end side can be suppressed.

### Fourth Embodiment

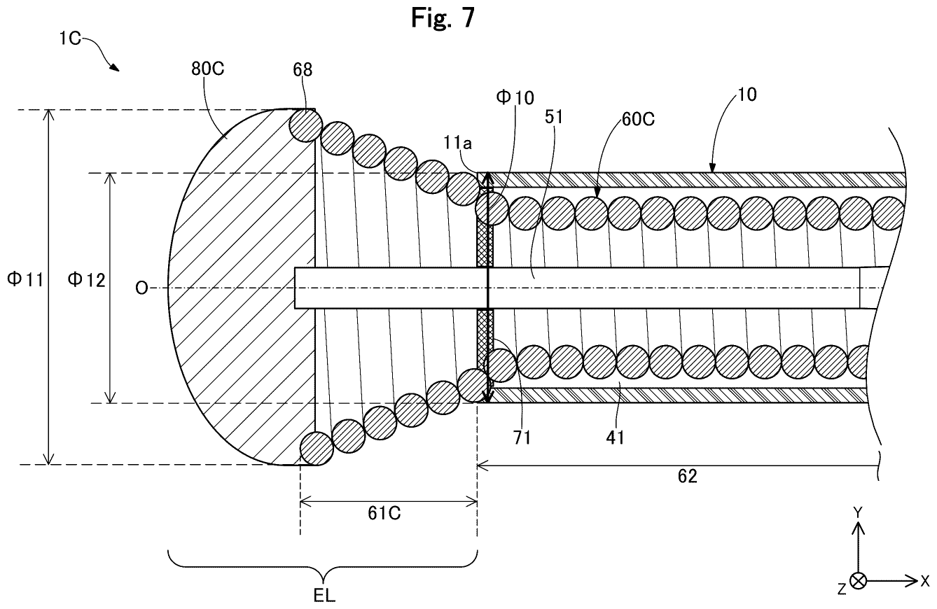

FIG. 7 is an enlarged sectional view illustrating a part of a guide wire 1C on the distal end side, according to the fourth embodiment. The guide wire 1C according to the fourth embodiment includes a coil body 60C instead of the coil body 60 and a distal tip 80C instead of the distal tip 80 in the configuration described in the first embodiment.

The coil body 60C includes a protruding portion 61C instead of the protruding portion 61. The protruding portion 61C has, in whole, a tapered shape with a diameter gradually decreasing from the distal end toward the proximal end. An outer diameter Φ11 of the distal end of the protruding portion 61C is larger than an outer diameter Φ12 of the proximal end of the protruding portion 61C. The outer diameter Φ11 of the distal end of the protruding portion 61C is larger than the outer diameter Φ10 of the distal ends of the insulating tubes 10, 20, and 30 (specifically, the outer diameter Φ10 of the distal end 11a of the first tube 10). On the other hand, the outer diameter Φ12 of the proximal end of the protruding portion 61C is equal to the outer diameter Φ10 of the distal ends of the insulating tubes 10, 20, and 30 (specifically, the outer diameter Φ10 of the distal end 11a of the first tube 10). The distal tip 80C has the maximum outer diameter (i.e. the outer diameter of the proximal end portion of the distal tip 80C) substantially equal to the outer diameter Φ11 of the distal end of the protruding portion 61C.

As described above, the configuration of the coil body 60C can be variously modified, and the coil body 60C may be configured such that the protruding portion 61C and the distal tip 80C (electrode portion EL) have an outer diameter larger than the outer diameter Φ10 of the first tube 10. Also, the guide wire 1C according to the fourth embodiment as described above can exhibit the same effect as in the aforementioned first embodiment.

According to the configuration of the fourth embodiment, since the protruding portion 61C has a tapered shape with an outer diameter gradually decreasing from the distal end toward the proximal end and the outer diameter Φ11 of the distal end of the protruding portion 61C is larger than the outer diameter Φ10 of the distal end 11a of the first tube 10, the surface area of the protruding portion 61C can be increased, i.e. the surface area of the electrode portion EL can be increased. As a result, the usability and safeness of the guide wire 1C can be further improved. According to the configuration of the fourth embodiment, since the outer diameter Φ12 of the proximal end of the protruding portion 61C is equal to the outer diameter Φ10 of the distal end 11a of the first tube 10, there is no step on the outer surface of the guide wire 1C on the distal end side, and the guide wire 1C can be prevented from being caught in the blood vessel or caught by other devices used in combination. Furthermore, fracture of the coil body 60C due to the step on the outer surface of the guide wire 1C on the distal end side can be suppressed.

### Fifth Embodiment

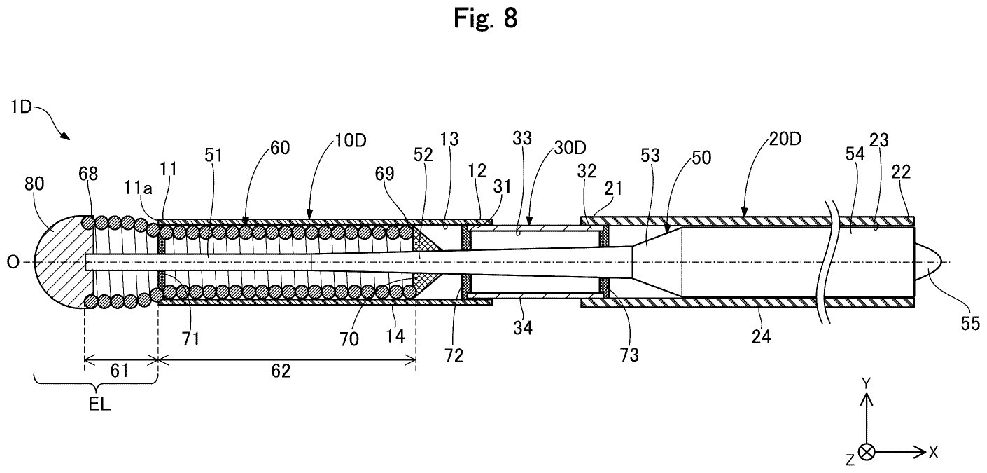

FIG. 8 is an explanatory view illustrating a sectional configuration of a guide wire 1D according to the fifth embodiment. The guide wire 1D according to the fifth embodiment includes a first tube 10D instead of the first tube 10, a second tube 20D instead of the second tube 20, and a third tube 30D instead of the third tube 30 in the configuration described in the first embodiment.

The first tube 10D is fixed in a state that the inner peripheral surface 13 of the first tube 10D is in contact with the outer peripheral surface of the covered portion 62 of the coil body 60. Similarly, the second tube 20D is fixed in a state that the inner peripheral surface 23 of the second tube 20D is in contact with the outer peripheral surface of the large diameter portion 54 of the core shaft 50. The third tube 30D is provided between the first tube 10D and the second tube 20D and fixed to the first tube 10D and the second tube 20D. As illustrated in FIG. 8, in the guide wire 1D, the first tube 10D and the covered portion 62 are in contact with each other, and the second tube 20D and the large diameter portion 54 are in contact with each other, and therefore the gas layers 41 and 42 described in the first embodiment are not formed.

As described above, the configuration of the guide wire 1D can be variously modified, and the gas layers need not be formed inside the insulating tubes 10, 20, and 30. Also, the guide wire 1D according to the fifth embodiment as described above can exhibit the same effect as in the aforementioned first embodiment. In the guide wire 1D according to the fifth embodiment, the diameter of the guide wire 1D can be decreased.

### Sixth Embodiment

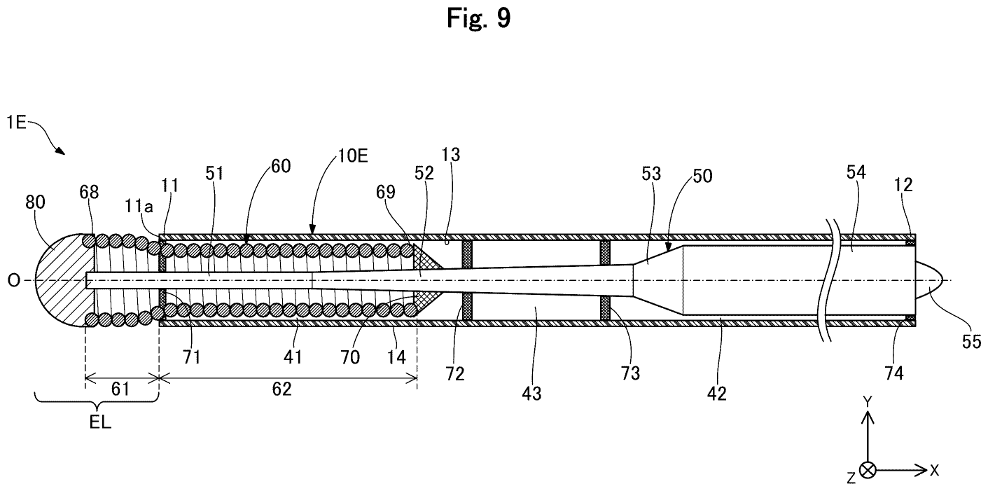

FIG. 9 is an explanatory view illustrating a sectional configuration of a guide wire 1E according to the sixth embodiment. The guide wire 1E according to the sixth embodiment includes a single insulating tube 10E instead of the insulating tubes 10, 20, and 30 in the configuration described in the first embodiment. The insulating tube 10E is a hollow cylindrical tubular body made of an insulating resin, which extends from the proximal end side with respect to the distal tip 80 and the protruding portion 61 to the proximal end portion of the large diameter portion 54.

As described above, the configuration of the guide wire 1E can be variously modified, and the guide wire 1E may be insulated by the single insulating tube 10E. In this case, the second fixation portion 72 and the third fixation portion 73 may be omitted. Although a case using the single insulating tube 10E has been described as an example in FIG. 9, the guide wire 1E may be insulated using two or four or more tubes. Also, the guide wire 1E according to the sixth embodiment as described above can exhibit the same effect as in the aforementioned first embodiment.

The disclosed embodiments have an object to improve the flexibility of a distal end portion of a guide wire having an electrode portion on its distal end.
(1) According to an aspect of the disclosed embodiments, a guide wire is provided. This guide wire includes a conductive core shaft, a conductive coil body surrounding a distal end portion of the core shaft, a conductive distal tip joined to a distal end of the core shaft and a distal end of the coil body, and an insulating tube made of a resin and covering a proximal end portion the coil body and a proximal end portion of the core shaft. The coil body has a covered portion covered by the insulating tube, and a protruding portion protruding from a distal end of the insulating tube toward the distal tip. The protruding portion of the coil body and the distal tip constitute an electrode portion of the guide wire for plasma ablation.

According to this configuration, the coil body has a distal end protruding portion protruding from the distal end of the insulating tube toward the distal tip, and the protruding portion of the coil body and the distal tip constitute the electrode portion for plasma ablation. That means, according to this configuration, since a surface area of the electrode portion is a sum of a surface area of the coil body protruding portion and a surface area of the distal tip (i.e. the surface area of the coil body protruding portion can be added to the surface area of the electrode portion), the surface area of the electrode portion can be easily increased compared to the conventional configurations with an electrode portion consisting only of a distal tip. If the electrode portion consists only of a distal tip, the distal tip needs to be enlarged for increasing the surface area of the electrode portion, which may compromise the flexibility of the distal end portion of the guide wire. According to this configuration, since the surface area of the electrode portion is a sum of the surface area of the coil body protruding portion and the surface area of the distal tip, the distal tip need not be excessively enlarged, and there is no risk of compromising the flexibility of the distal end portion of the guide wire. Furthermore, the protruding portion has a coil shape, and therefore, even if the distal end portion (electrode portion) of the guide wire collides with a blood vessel wall, the impact can be relieved by the protruding portion, and damage to the blood vessel wall can be suppressed. As a result, in a guide wire having an electrode portion on its distal end, the flexibility of the distal end portion and the safeness of the procedure can be improved.
(2) The guide wire according to the above aspect may be configured such that the protruding portion has a distal end straight portion having a constant outer diameter, and a proximal end tapered portion having an outer diameter gradually decreasing from the distal end of the tapered portion to the proximal end of the tapered portion.

According to this configuration, since the protruding portion of the coil body has the distal end straight portion having a constant outer diameter, and the proximal end tapered portion having an outer diameter gradually decreasing from the distal end of the coil body toward the proximal end of the coil body, the surface area of the protruding portion of the coil body, i.e. the surface area of the electrode portion can be increased. Since the increase in the surface area of the electrode portion can also contribute to improvement in visibility of the electrode portion in an X-ray image (angiogram), the usability of the guide wire can be improved. Also, the increase in the surface area of the electrode portion leads to reduction in the risk of blood vessel perforation, and therefore the safeness of the guide wire can be improved.
(3) The guide wire according to the above aspects may be configured such that the outer diameter of the proximal end of the straight portion of the protruding portion of the coil body is equal to the outer diameter of the distal end of the insulating tube.

According to this configuration, since the outer diameter of the proximal end of the straight portion of the protruding portion of the coil body is equal to the outer diameter of the distal end of the insulating tube, the outer diameter of the entire distal end side of the guide wire (specifically, the electrode portion of the guide wire, excluding the tapered portion of the protruding portion of the coil body, and the insulating tube) can be made constant. As a result, the guide wire can be prevented from being caught in the blood vessel or caught by other devices used in combination.
(4) The guide wire according to the above aspects may be configured such that the protruding portion of the coil body has a constant first outer diameter, the covered portion of the coil body has a constant second outer diameter smaller than the first outer diameter, and the coil body includes, between the protruding portion of the coil body and the covered portion of the coil body, a stepped portion of the coil body where an outer diameter of the coil body changes from the first outer diameter of the protruding portion of the coil body to the second outer diameter of the covered portion of the coil body.

According to this configuration, since the protruding portion of the coil body has a constant first outer diameter and the first outer diameter of the protruding portion of the coil body is larger than the second outer diameter of the covered portion of the coil body, the surface area of the protruding portion of the coil body can be increased, i.e. the surface area of the electrode portion of the guide wire can be increased. As a result, the usability and the safeness of the guide wire can be further improved.
(5) The guide wire according to the above aspects may be configured such that the protruding portion of the coil body has a tapered shape with an outer diameter gradually decreasing from the distal end of the protruding portion of the coil body toward the proximal end of the protruding portion of the coil body, and the outer diameter of the distal end of the protruding portion of the coil body is larger than an outer diameter of the distal end of the insulating tube.

According to this configuration, since the protruding portion of the coil body has a tapered shape with an outer diameter gradually decreasing from the distal end of the protruding portion of the coil body toward the proximal end of the protruding portion of the coil body and the outer diameter of the distal end of the protruding portion of the coil body is larger than the outer diameter of the distal end of the insulating tube, the surface area of the protruding portion of the coil body can be increased, i.e. the surface area of the electrode portion of the guide wire can be increased. As a result, the usability and the safeness of the guide wire can be further improved.
(6) The guide wire according to the above aspects may be configured such that the outer diameter of the proximal end of the protruding portion of the coil body is equal to the outer diameter of the distal end of the insulating tube.

According to this configuration, since the outer diameter of the proximal end of the protruding portion of the coil body is equal to the outer diameter of the distal end of the insulating tube, the outer diameter of the entire distal end side of the guide wire (specifically, the electrode portion of the guide wire and the insulating tube) can be made constant. As a result, the guide wire can be prevented from being caught in the blood vessel or caught by other devices used in combination.
(7) The guide wire according to the above aspects may be configured such that the electrode portion of the guide wire is a return electrode.

According to this configuration, since the electrode portion of the guide wire is a return electrode, the guide wire can be configured as a so-called return guide wire that is used in combination with a plasma guide wire.
(8) According to an aspect of the disclosed embodiments, a medical system is provided. This medical system includes the guide wire according to the above aspects, a plasma guide wire having a distal end electrode, and a high frequency generator, in which, when a high frequency wave is applied to the guide wire and the plasma guide wire from the high frequency generator, the electrode portion of the guide wire serves as a return electrode that generates a plasma on the distal end electrode of the plasma guide wire.

According to this configuration, it is possible to provide a medical system including a plasma guide wire and a guide wire (so-called return guide wire) used in combination with the plasma guide wire.

The disclosed embodiments can be embodied according to various aspects, and, for example, in a form of a guide wire, a plasma guide wire, a medical system including the guide wire and the plasma guide wire, and a production method therefor.

### MODIFICATIONS OF EMBODIMENTS

The disclosed embodiments are not limited to the above-described embodiments and may be implemented in various modes without departing from the gist thereof, and for example, the following modifications are also possible.

### Modification 1

Examples of the configuration of the medical system 1000 have been described in the first to sixth embodiments above. However, the configuration of the medical system 1000 can be variously modified. For example, the configuration of the plasma guide wire 100 described in FIG. 1 is merely an example, and variously modified. For example, instead of the first to third tubes 110 to 130, one or any number of tubes may be used, and the gas layers 141 to 143 may be omitted. In FIG. 1 and FIG. 4, a case using the guide wires 1 and 1A to 1E as so-called return guide wires has been described as an example. However, the guide wires 1 and 1A to 1E may be used as plasma guide wires that generate a plasma on the electrode portion EL. In other words, the guide wires 1 and 1A to 1E may be configured to play the role of the plasma guide wire 100 described in FIG. 1 and FIG. 4.

### Modification 2

In the first to sixth embodiments, examples of the configurations of the guide wires 1 and 1A to 1E have been described. However, the configurations of the guide wires 1 and 1A to 1E can be variously modified. For example, the first tube 10, the second tube 20, and the third tube 30 may be integrally formed. For example, the core shaft 50 is not limited to the above-described shape but may have any shape. For example, at least a part of the small diameter portion 51, the first tapered portion 52, the second tapered portion 53, the large diameter portion 54, and the proximal end portion 55 described as examples in the above embodiments may be omitted. For example, the guide wire may include additional configurations not described above. For example, an inner coil body may be provided inside the coil body 60. For example, a protective member for protecting the first tube 10 from the electric discharge may be provided on the distal end portion of the first tube 10. For example, a protective member for protecting the joint part may be provided between the first tube 10 and the third tube 30 or between the third tube 30 and the second tube 20. For example, a color marker for improving visibility in visual observation or a radiopaque marker for improving visibility in X-ray images may be provided on the distal end 11a of the first tube 10 or at any position.

### Modification 3

The configurations of the guide wires 1 and 1A to 1E according to the first to sixth embodiments, and the configurations of the guide wires 1 and 1A to 1E according to Modifications 1 and 2 above may be combined as appropriate. For example, the guide wire 1D described in the fifth embodiment may be configured to include the coil bodies 60A, B, and C described in any of the second, third, and fourth embodiments. For example, in the guide wire 1E described in the sixth embodiment may be configured to include the coil bodies 60A, B, and C described in any of the second, third, and fourth embodiments.

Although the present aspects have been explained above based on the embodiments and the modifications, the embodiments of the above-described aspects are intended to facilitate understanding of the present aspects and do not limit the present aspects. The present aspects may be modified and improved without departing from the gist and the scope of claims and include equivalents thereof. Unless the technical features are described as essential in the present specification, the technical features may be deleted as appropriate.

### DESCRIPTION OF REFERENCE NUMERALS

1, 1A to 1E . . . Guide wire
10, 10D, 10E . . . First tube (insulating tube)
20, 20D . . . Second tube (insulating tube)
30, 30D . . . Third tube (insulating tube)
41, 42, 43 . . . Gas layer
50 . . . Core shaft
51 . . . Small diameter portion
52 . . . First tapered portion
53 . . . Second tapered portion
54 . . . Large diameter portion
55 . . . Proximal end portion
60, 60A to 60C . . . Coil body
61, 61A to 61C . . . Protruding portion
62, 62B . . . Coated portion
63 . . . Stepped portion
70 . . . Coil fixation portion
71, 71B . . . First fixation portion
72 . . . Second fixation portion
73 . . . Third fixation portion
74 . . . Fourth fixation portion
80, 80A, 80C . . . Distal tip
82 . . . Distal end side joint part
83 . . . Proximal end side joint part
100 . . . Plasma guide wire
110 . . . First tube
120 . . . Second tube
130 . . . Third tube
141, 142, 143 . . . Gas layer
150 . . . Core shaft
160 . . . Coil body
170 . . . Coil fixation portion
171 . . . First fixation portion
172 . . . Second fixation portion
173 . . . Third fixation portion
174 . . . Fourth fixation portion
180 . . . Distal tip
200 . . . RF generator
210 . . . First terminal
211 . . . First cable
220 . . . Second terminal
221 . . . Second cable
300 . . . Catheter
301 . . . First shaft
302 . . . Second shaft
303 . . . Distal tip
611 Straight portion
612 . . . Tapered portion
1000 . . . Medical system

What is claimed is:

1. A guide wire comprising:
a conductive core shaft;
a conductive coil body surrounding a distal end portion of the core shaft;
a conductive distal tip joined to a distal end of the core shaft and a distal end of the coil body; and
an insulating tube made of a resin and covering a proximal end portion of the coil body and a proximal end portion of the core shaft,
the coil body having a covered portion covered by the insulating tube and a protruding portion protruding from a distal end of the insulating tube toward the distal tip,
wherein the protruding portion of the coil body and the distal tip constitute an electrode portion of the guide wire for plasma ablation.

2. The guide wire according to claim 1, wherein
the protruding portion of the coil body has:
a straight portion having a constant outer diameter, and
a tapered portion disposed on a proximal end side with respect to the straight portion and having an outer diameter gradually decreasing from a distal end of the tapered portion to a proximal end of the tapered portion.

3. The guide wire according to claim 2, wherein
the outer diameter of a proximal end of the straight portion is equal to an outer diameter of the distal end of the insulating tube.

4. The guide wire according to claim 1, wherein
the protruding portion has a constant first outer diameter,
the covered portion has a constant second outer diameter smaller than the first outer diameter, and
the coil body comprises, between the protruding portion and the covered portion, a stepped portion where an outer diameter of the coil body changes from the first outer diameter to the second outer diameter.

5. The guide wire according to claim 1, wherein
the protruding portion has a tapered shape with an outer diameter gradually decreasing from a distal end toward a proximal end, and
the outer diameter of the distal end of the protruding portion is larger than an outer diameter of the distal end of the insulating tube.

6. The guide wire according to claim 4, wherein
the outer diameter of the proximal end of the protruding portion is equal to the outer diameter of the distal end of the insulating tube.

7. The guide wire according to claim 1, wherein
the electrode portion is a return electrode.

8. The guide wire according to claim 5, wherein
the outer diameter of the proximal end of the protruding portion is equal to the outer diameter of the distal end of the insulating tube.

9. The guide wire according to claim 2, wherein
the electrode portion is a return electrode.

10. The guide wire according to claim 3, wherein
the electrode portion is a return electrode.

11. The guide wire according to claim 4, wherein
the electrode portion is a return electrode.

12. The guide wire according to claim 5, wherein
the electrode portion is a return electrode.

13. The guide wire according to claim 6, wherein
the electrode portion is a return electrode.

14. A medical system comprising:
the guide wire according to claim 1; and
a plasma guide wire having a distal end electrode, wherein
when a high frequency wave is applied to the guide wire and the plasma guide wire from a high frequency generator, the electrode portion of the guide wire serves as a return electrode that generates a plasma on the distal end electrode of the plasma guide wire.
